(19) United States
(12) Patent Application Publication
(10) Pub. No.: US 2009/0105543 A1
(43) Pub. Date: Apr. 23, 2009

(54) Endoscope Lens Cleaner

(76) Inventors: Eric C. Miller, Los Gatos, CA (US); Greg Schmitz, Los Gatos, CA (US); Scott M. Smith, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/253,089

(22) Filed: Oct. 16, 2008

Related U.S. Application Data
(60) Provisional application No. 60/981,347, filed on Oct. 19, 2007.

(51) Int. Cl. A61B 1/12; A61M 5/00

(52) U.S. Cl. 600/155; 604/257

(57) ABSTRACT

A device for cleaning a lens of an endoscope inside a patient during a surgical procedure may include a cannula having a proximal end, a distal end and an opening, and an expandable member coupled with the cannula such that when fluid is introduced into the expandable member, it expands out of the opening in the cannula. A method for cleaning a lens of an endoscope inside a patient during a surgical procedure may involve: advancing a cannula into the patient; passing fluid into an expandable member coupled with the cannula to expand the expandable member, wherein some of the fluid passes through the expandable member to an outer surface of the expandable member; and contacting the endoscope lens with the outer surface of the expandable member, located inside the patient, to clean the lens.

### CROSS-REFERENCES TO RELATED APPLICATIONS

This application claims priority to U.S. Provisional Patent Application Ser. No. 60/981,347 filed on Oct. 19, 2007; the full disclosure of which is hereby incorporated by reference.

### BACKGROUND OF THE INVENTION

1. Field of the Invention

The present invention relates generally to the construction and use of percutaneous access devices in performing laparoscopic and related endoscopic surgical procedures. More particularly, the present invention relates to a device and method for in situ cleaning of laparoscopes and other viewing scopes used in such surgical procedures.

Laparoscopic and other endoscopic surgical procedures rely on percutaneous introduction of a viewing scope into an internal region within the patient where the surgical procedure is to be performed. In the case of laparoscopic procedures, the viewing scope is commonly referred to as an endoscope or a laparoscope, and the laparoscope is commonly introduced through an access tube, such as a trocar, which passes into the patient's abdomen. The abdomen will have been insufflated to provide a working region. Using a laparoscope introduced through a trocar, the surgeon can view the region to be treated on a video monitor and can perform a variety of surgical procedures using specialized surgical instruments which are introduced percutaneously either directly or through trocars or other access tubes. Exemplary procedures which may be performed laparoscopically include cholecystectomy, hysterectomy, gastrostomy, appendectomy, bowel resection, herniorrhaphy, and the like. Analogous surgical procedures may be performed elsewhere in the body using other conventional viewing scopes, such as endoscopes, arthroscopes, thoracoscopes, bronchioscopes, hysteroscopes, choledochoscopes, cystoscopes, resectoscopes, and the like.

In all such procedures that employ internally-introduced viewing scopes, problems can arise with fogging and fouling of the distal lens of the scope, which provides the optical access. The most common approach for dealing with such obscuring of the distal lens has been to remove the viewing scope from the patient and manually clean it. For example, commercial products are available comprising a sponge or fabric pad and a bottle of cleaning solution. The surgeon can saturate the sponge with the cleaning solution and clean the distal lens by removing the viewing scope from the patient, wiping the distal lens against the sponge, and returning the viewing scope to the patient. While this removal and manual wiping process works to temporarily clean the endoscope lens, the need to withdraw the viewing scope from the patient, clean it, reinsert it, and relocate the target is highly inefficient and inconvenient. Furthermore, it is often the case that the entry port into the patient may collect blood or other debris so that simply advancing the distal end of the scope back into the patient through the port immediately gets the scope lens dirty again. Thus, just keeping the lens of an endoscope clean enough to enable a laparoscopic procedure is often quite inconvenient and extends the operative time for performing such a procedure.

It has also been proposed to incorporate a spray wash nozzle on the viewing scope itself in order to permit cleaning of a distal lens without removing the scope from the patient. While addressing the needs of efficiency, the requirement of incorporating a washing system in the viewing scope itself does not permit cleaning of existing viewing scopes which are already in use. Such viewing scopes can be relatively expensive, limiting the ability to replace such scopes with models incorporating a wash system. Moreover, incorporation of at least one additional lumen and associated hardware for the wash system further complicates construction of the viewing scope, making it more expensive and requiring a larger diameter. Additionally, washing of a distal lens by simply spraying it with liquid while the viewing scope is in place will not always be effective in cleaning the lens. Many times, it will still be necessary to withdraw the viewing scope to actually wipe the lens clean.

For these reasons, it would be desirable to provide alternative devices and methods for cleaning surgical viewing scopes in situ, i.e., without the need to remove the viewing scope from the patient. Such devices and methods should not require the modification of the viewing scope in any manner, and should preferably require minimum or no modification of other instruments used in performing the surgical procedure, e.g., trocars used for introducing the viewing scope. Such devices and methods should be very effective in removing contaminating debris and fogging of the distal lens of the viewing scope, should be convenient to use, and should be low-cost to implement. Some or all of these objectives will be met by the various embodiments of the present invention described hereinafter.

2. Description of the Background Art

U.S. Pat. No. 5,514,084 describes a wipe for cleaning the lens at the forward end of an endoscope while it is inside a body cavity during endoscopic surgery. PCT Patent Application Pub. No. WO 2008/041225A2 describes an umbrella-like endoscope lens cleaning device. U.S. Pat. Nos. 4,281,646 and 4,919,113 and Japanese Patent 5-103751 describe lens cleaning devices involving squirting liquid at the lens of an endoscope. U.S. Pat. No. 5,274,874 describes a sponge device for cleaning an endoscope. U.S. Pat. Nos. 4,281,646; 5,313,934; 5,400,767; and 6,354,992 describe various endoscope cleaning devices involving sheaths, tubes or the like that are coupled with endoscopes.

U.S. Pat. No. 4,656,999 describes a contact endoscope having a slidable blade for severing tissue at its distal end. EP 497 347 describes a laparoscope having a lens washing nozzle at its distal end. Scope washing systems are also described in U.S. Pat. Nos. 5,207,213; 4,841,952; 4,760,838; and 4,646,722. U.S. Pat. No. 4,684,874 describes a dissolvable membrane which covers an endoscope lens to protect the lens while being inserted into a patient. U.S. Pat. No. 4,682,585 discloses an endoscope having annular depressions intended to remove contaminants while passing through a trocar. U.S. Pat. No. 4,919,113 describes a spray cleaner for an endoscope. U.S. Pat. No. 4,177,814 describes a self-sealing cannula having an elastomeric valve at its proximal end. Dexide, Fort Worth, Tex., sells a fog reduction/elimination device (FRED) which comprises a sponge and a bottle of cleaning solution, where a scope is removed from the patient to permit cleaning by the sponge. U.S. Pat. Nos. 5,127,909; 5,053,016; and 4,943,280 describe trocar assemblies having flapper valves at their proximal ends for sealing against insufflation pressure. A radially expanding dilator which can incorporate the cleaning assembly of the present invention is described in U.S. Pat. No. 5,183,464.

### BRIEF SUMMARY OF THE INVENTION

The present invention is generally directed to a device, method and system for cleaning an endoscope lens in situ—i.e., while the lens is inside a patient. In many embodiments, the cleaner is percutaneous, meaning it is inserted through the patient's skin. In some embodiments, the cleaner may be inserted directly through the skin using a piercing device or member coupled with the cleaning device. Alternatively, in some embodiments, the cleaner may be inserted through an entry port into the patient that has been placed previously. The various aspects of the invention are described further below.

In one aspect of the present invention, a device for cleaning a lens of an endoscope inside a patient during a surgical procedure may include a cannula having a proximal end, a distal end and an opening, and an expandable member coupled with the cannula such that when fluid is introduced into the expandable member, it expands out of the opening in the cannula. In some embodiments, the cannula may have a sharp distal tip for piercing through skin, and the sharp distal tip may be convertible into a blunt tip after it pierces through the skin. In one embodiment, the cannula may comprise a varies needle. In another embodiment, the device may further include a piercing member disposed in the cannula and having a sharp tip for piercing through skin, with the piercing member being slidably removable from the cannula.

In some embodiments, the cannula may have an outer diameter of 5 mm or less. Optionally, the device may also include a skin anchor member slidably coupled with an outer surface of the cannula for anchoring the cannula to the patient's skin during the surgical procedure. In some embodiments, the expandable member may comprise a balloon. Optionally, the balloon may be at least semi-permeable. In some embodiments, the balloon may include multiple pores for allowing leakage of inflation fluid onto an outer surface of the balloon. In some embodiments, the device may include a tube coupled with the balloon and extending through the cannula to the proximal end of the cannula to provide a means for delivering expansion fluid to the balloon. In an alternative embodiment, the expandable member may comprise a sponge or other fluid absorbing material. In one embodiment, the fluid absorbing material may be at least partially covered or surrounded by an expandable nitinol wire cage.

In some embodiments, the opening at or near the distal end of the cannula may be in a side wall of the cannula. Alternatively, the opening may be in an extreme distal end of the cannula. In one embodiment, an outer surface of the expandable member may have a white color to facilitate white balancing of an endoscope device in situ. In this or other embodiments, the outer surface may further include at least one of letters or symbols to facilitate focusing of the endoscope device in situ.

In another aspect of the present invention, a device for cleaning a lens of an endoscope inside a patient during a surgical procedure may include a cannula having a proximal end, a distal end and an opening in a side wall of the cannula. The device may further include a lens cleaning member disposed inside the cannula such that a cleaning portion of the lens cleaning member is exposed through the opening in the cannula. In one embodiment, the lens cleaning member may include at least one wiper blade for cleaning an endoscope lens. Some embodiments may include multiple wiper blades. Some embodiments may include one or more fluid ports near the wiper blade(s) for delivering lens cleaning fluid to the blades or an area near the blades.

In another aspect of the present invention, a system for cleaning a lens of an endoscope inside a patient during a surgical procedure may include: a cannula having a proximal end, a distal end and an opening; an expandable member coupled with the cannula such that when fluid is introduced into the expandable member it expands out of the opening in the cannula; a fluid delivery tube coupled with the expandable member and extending through the cannula to an opening at or near the proximal end of the cannula; and an amount of cleaning fluid for passing through the fluid delivery tube and expanding the expandable member and for cleaning the endoscope lens.

In one embodiment, the system may further include a syringe, and the cleaning fluid may be housed in the syringe. In one embodiment, the system may further include a piercing member slidably disposed within the cannula and having a sharp distal tip. In various embodiments, the fluid may be, but is not limited to, saline solution, glycol solution, water, alcohol, contact lens cleaning solution and/or combinations thereof. In some embodiments, the expandable member may comprise a balloon, and the balloon may include multiple pores through which the cleaning fluid leaks from inside the balloon to an outer surface of the balloon.

In another aspect of the invention, a method for cleaning a lens of an endoscope inside a patient during a surgical procedure may involve: advancing a cannula into the patient; passing fluid into an expandable member coupled with the cannula to expand the expandable member, wherein some of the fluid passes through the expandable member to an outer surface of the expandable member; and contacting the endoscope lens with the outer surface of the expandable, located inside the patient, to clean the lens.

In some embodiments, passing the fluid into the expandable member may involve passing fluid through an inflation tube into a balloon. In some embodiments, advancing the needle may involve advancing the needle through skin of the patient using a sharp distal tip and converting the sharp distal tip to a blunt tip. In some embodiments, the method may further include anchoring the cannula to the patient's skin using a skin anchor coupled with the cannula. In some embodiments, the method may further include white balancing the endoscope inside the patient, during the procedure, using a white-colored outer surface of the expandable member. Such a method may also further involve focusing the endoscope inside the patient, during the procedure, using at least one of symbols or letters disposed on an outer surface of the balloon.

These and other aspects and embodiments are described more fully below in the Detailed Description, with reference to the attached Drawings.

### BRIEF DESCRIPTION OF THE DRAWINGS

FIG. 1 is cross-sectional view of a patient's insufflated abdomen and a side view of an endoscope cleaning device with an endoscopic lens pressed against the outside of the device, according to one embodiment of the present invention;

FIGS. 2A-2D demonstrate a method for inserting and inflating an endoscopic lens cleaning device, according to one embodiment of the present invention;

FIG. 3 is cross-sectional view of a patient's insufflated abdomen and a side view of an endoscope cleaning device with an endoscopic lens pressed against the outside of the device, according to an alternative embodiment of the present invention;

FIGS. 4A-4D demonstrate a method for inserting a scope cleaner into a body cavity, according to one embodiment of the present invention;

FIGS. 5A-5D are cross-sectional, perspective, exploded and perspective views, respectively, of a scope cleaner having a side-facing opening with an expandable member, according to one embodiment of the present invention;

FIGS. 6A and 6B are cross-sectional and perspective views, respectively, of a scope cleaner having an expandable member disposed proximal of the device distal end, according to one embodiment of the present invention;

FIGS. 7A and 7B are exploded and perspective views, respectively, of a scope cleaner device having an expandable member at its distal end, according to one embodiment of the present invention;

FIGS. 8A-8D are cross-sectional, perspective, end-on and perspective views, respectively, of a scope cleaner device having an expandable wire member at its distal end, according to one embodiment of the present invention;

FIGS. 9A-9D are cross-sectional, end-on, exploded and perspective views, respectively, of an endoscope cleaner device having one or more wiper blades, according to one embodiment of the present invention;

FIG. 10 is a perspective view of an endoscope cleaner system, according to one embodiment of the present invention;

FIG. 11 is a perspective view of an endoscope cleaner, according to one embodiment of the present invention;

FIG. 12 is a perspective view of an endoscope cleaner, according to an alternative embodiment of the present invention;

FIG. 13 is a perspective view of a balloon portion of an endoscope cleaner, according to one embodiment of the present invention;

FIG. 14 is a perspective view of a balloon portion of an endoscope cleaner, according to another embodiment of the present invention;

FIGS. 15A and 15B are perspective and side views, respectively, of an endoscope cleaner having a concave, sail-shaped cleaning portion, according to one embodiment of the present invention; and

FIG. 16 is a side view of an endoscope cleaner having a sponge distal portion, according to one embodiment of the present invention.

### DETAILED DESCRIPTION OF THE INVENTION

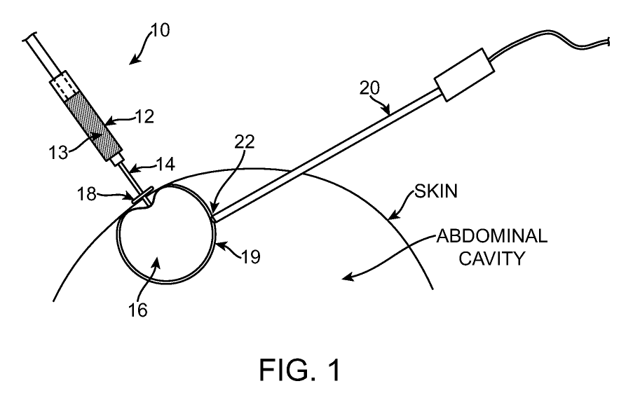

Referring to FIG. 1, in one embodiment, an endoscope lens cleaner device 10 may include a cannula 14, attachable to a syringe 12 for dispensing a fluid 13, a slidable stop member 18 coupled with cannula 14, and a balloon 16 coupled with cannula 14. In various embodiments, balloon 16 may be semi-permeable, permeable or non-permeable. Fluid 13, which may be saline solution, glycol solution, alcohol solution, water, some combination thereof, or any other biocompatible fluid capable of cleaning an endoscope lens, may be passed into balloon 16 via syringe 12 (or via other fluid introduction devices in alternative embodiments) such that it inflates balloon 16 and also slowly seeps out of balloon 16, forming a thin fluid layer 19 on the outer surface of balloon 16. An endoscope 20 residing partially in the patient may then be manipulated to contact an endoscope lens 22 with the outer surface of balloon 16 and fluid layer 19 to clean endoscope lens 22. Stop member 18 may be used to anchor and/or support device 10 by sliding down cannula 14 to trap the patient's skin between stop member 18 and inflated balloon 16. By anchoring device 10 in this way, it may be possible to press and/or wipe endoscope lens 22 against the outer surface of balloon 16, which may facilitate the lens cleaning process.

In various embodiments, cannula 14 (which may alternatively be referred to as a “needle”) may have any of a number of different shapes, sizes and configurations. In some embodiments, for example, cannula 14 may comprise a stainless steel (or other metal or alternative rigid material) tube with an outer diameter of about 10 mm or less, and ideally about 5 mm or less. In some embodiments, cannula 14 may have a sharp tip, while in alternative embodiments, cannula 14 may have a dull tip and a piercing member may be used to help advance cannula 14 through a patient's skin. Balloon 16 may be made of any suitable permeable material, such as but not limited to a polymer. In some embodiments, balloon 16 may be configured to allow one or more fluids 13 to seep slowly through its surface to form fluid layer 19. In some embodiments, multiple pores or micropores may be formed in balloon 16 to allow for such seepage. In alternative embodiments, where balloon 16 is not permeable, it may be coated or impregnated with a lens cleaning fluid or substance. Again, any suitable fluid may be used, such as saline solution, contact fluid cleaner or the like. In one embodiment, cooled fluid may be introduced into and contained within balloon 16 and not allowed to seep through balloon 16. The cooled fluid may cause condensation to form on the outside surface of balloon 16, which condensation may be used to clean an endoscope lens. Such condensation may form readily, for example, when balloon 16 is located in a relatively warm environment such as an abdominal cavity.

In some embodiments, all or a portion of an outer surface of balloon 16 may have a white color or shade of white, to facilitate white balancing of an endoscope inside the patient during a surgical procedure. White balancing is usually performed before a surgical procedure and then cannot be performed again without removing the endoscope from the patient. Another optional feature of balloon 16 is to include one or more symbols and/or letters on the outer surface of balloon 16 to facilitate focusing of the endoscope inside the patient during a procedure. Again, focusing an endoscope typically requires removing the scope from the patient, so the ability to focus inside the patient during a procedure would be advantageous.

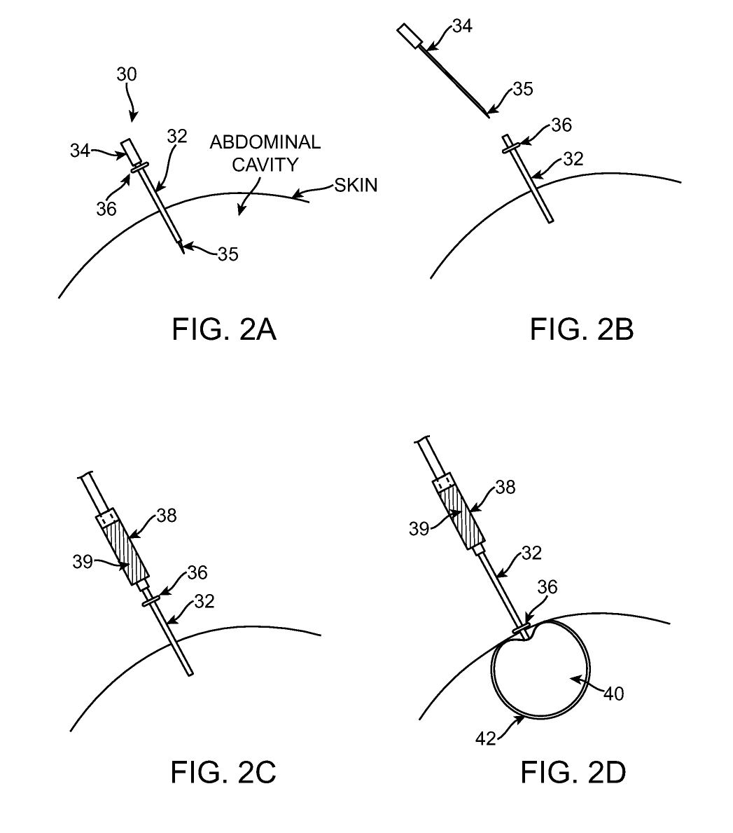

Referring to FIGS. 2A-2D, a method for inserting and inflating an endoscope lens cleaning device 30, according to one embodiment of the present invention, is shown. In FIG. 2A, a cannula 32 with a piercing member 34 disposed therein is advanced through the patient's skin, using a sharp distal tip 35 of piercing member 34 to pierce the skin. A stop member 36 on cannula 32 is in the retracted position. In FIG. 2B, cannula 32 is in place, and piercing member 34 is removed. In FIG. 2C, a syringe 38 containing a lens cleaning fluid 39 is coupled with one end of cannula 32. Next, as shown in FIG. 2D, fluid is introduced into a permeable balloon 40, coupled with cannula 32, so that balloon 40 inflates and fluid 39 seeps slowly through the surface of balloon 40 to form a fluid layer 42. When balloon 40 is partially or completely inflated, device 30 may be pulled back so that balloon 40 abuts the inner wall of the patient's insufflated abdomen, as shown in FIG. 2D. Stop member 36 may then be advanced, as shown, to anchor device 30 with the patient's skin between stop member 36 and balloon 40. In various embodiments, a combination of cannula 32 with stop member 36 and balloon 40, piercing member 34, syringe 38 and fluid 39 may be provided as a system or kit to a user. All these items or any subset thereof may be provided in such a kit.

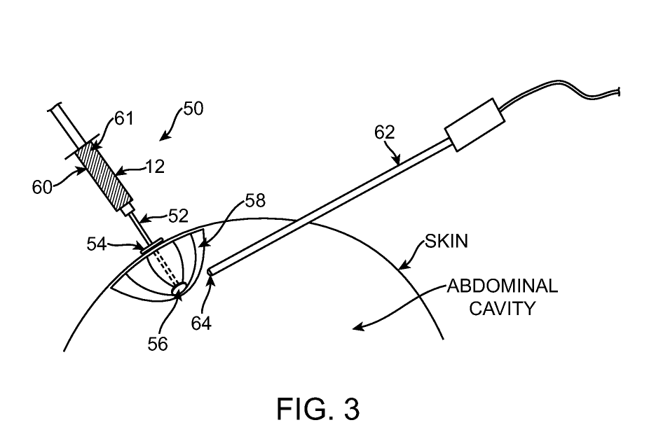

Referring now to FIG. 3, an alternative embodiment of an endoscope lens cleaner device 50 may include a cannula 52 with slidably attached stop member 54, a permeable material 56 coupled with cannula 52, and one or more drying materials 58 coupled with permeable material 56. In the configuration shown in FIG. 3, permeable material 56 and drying materials 58 together have a configuration similar to that of an umbrella and are also expandable and contractible like an umbrella. Device 50 may also include a syringe 60 and lens cleaning fluid 61 in some embodiments. As with the balloon embodiment, when fluid 61 is passed into permeable material 56 the material may expand and/or may allow fluid 61 to seep slowly through it to form a fluid layer on its outer surface. Alternatively, a fluid layer may not form in some embodiments, but permeable material 56 may simply remain wet with fluid 61, to provide a surface on which a lens 64 of and endoscope 62 may be cleaned. After lens 64 is cleaned via fluid 61 and permeable material 56, it may then be pressed or wiped against one or more pieces of drying material 58.

In various embodiments, permeable material 56 may be made of any suitable material, such as but not limited to sponge, cloth, surgical sponge, expandable polymeric materials and the like. Drying materials 58 may also include any suitable material for drying and/or wiping fluid off the surface of lens 64, such as Gortex, wicking material(s) and/or the like. In various embodiments, permeable material 56 and/or drying materials 58 may have a white or white-like color for facilitating endoscope 62 white balancing. Also in various embodiments, one or more symbols or letters, such as the word “FOCUS” as shown in FIG. 3, may be included on a surface of permeable material 56 and/or drying materials 58, to facilitate focusing of endoscope 62 inside the patient.

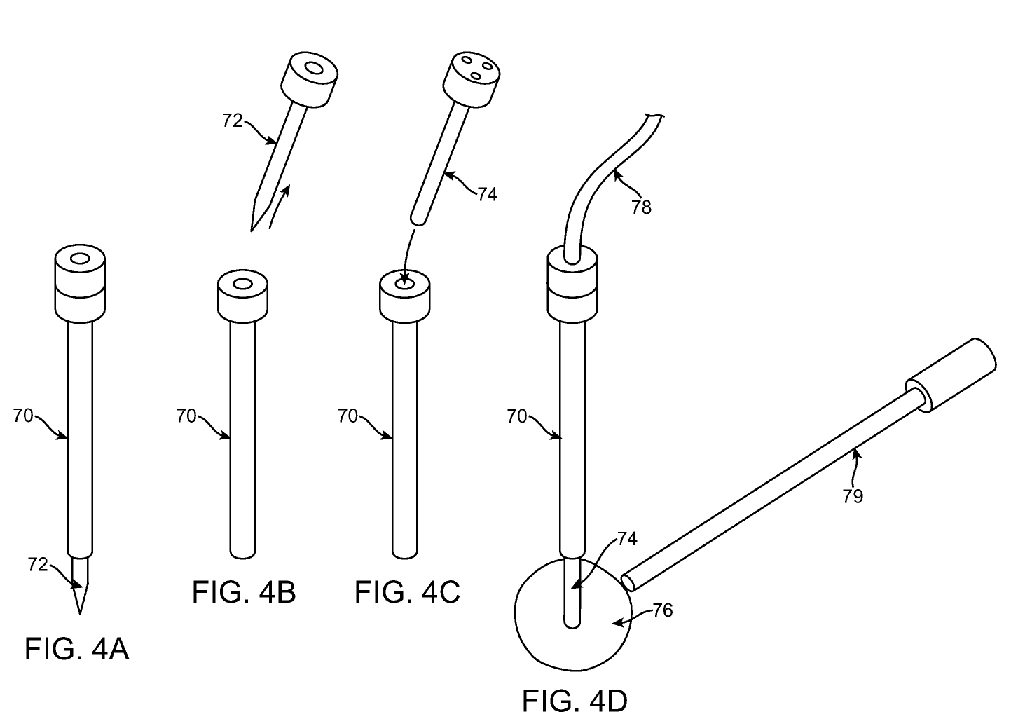

Referring now to FIGS. 4A-4D, one method for inserting an endoscope cleaning device into a body cavity is demonstrated. In various embodiments, an endoscope cleaner may either be inserted into a cavity through a preexisting opening, such as a laparoscopic port, or the cleaner device or system itself may be used to pierce into the body cavity to gain access. In the embodiment shown in FIGS. 4A-4D, an endoscope cleaning system itself is used to gain access.

In FIG. 4A, a cannula 70 is shown coupled with a trocar 72 having a sharp distal tip. In one method, the coupled cannula 70 and trocar 72 may be advanced into a body cavity such that the trocar 72 provides the piercing function and the distal tip of the cannula 70 is positioned in the body cavity. As shown in FIG. 4B, trocar 72 may then be removed from cannula 70, and as shown in FIG. 4C, an endoscope cleaning device 74 may then be inserted through cannula 70 to position its distal tip in the body cavity. FIG. 4D shows endoscope cleaning device 74 positioned through cannula 70, as it would be inside a body cavity, with a distal expandable member 76 expanded. In some embodiments, a fluid delivery device such as an irrigation tube 78 or syringe may be coupled with a proximal end of endoscope cleaning device 74 to provide fluid to expandable member 76. In many embodiments, the delivered fluid acts both to expand expandable member 76 and leaks or weeps slowly out of expandable member 76 to act as a cleaning fluid for an endoscope 79. Thus, as shown in FIG. 4D, endoscope 79 lens may be cleaned by touching, pressing against and/or wiping against an outer surface of expandable member 76, where typically there will be some amount of cleaning fluid.

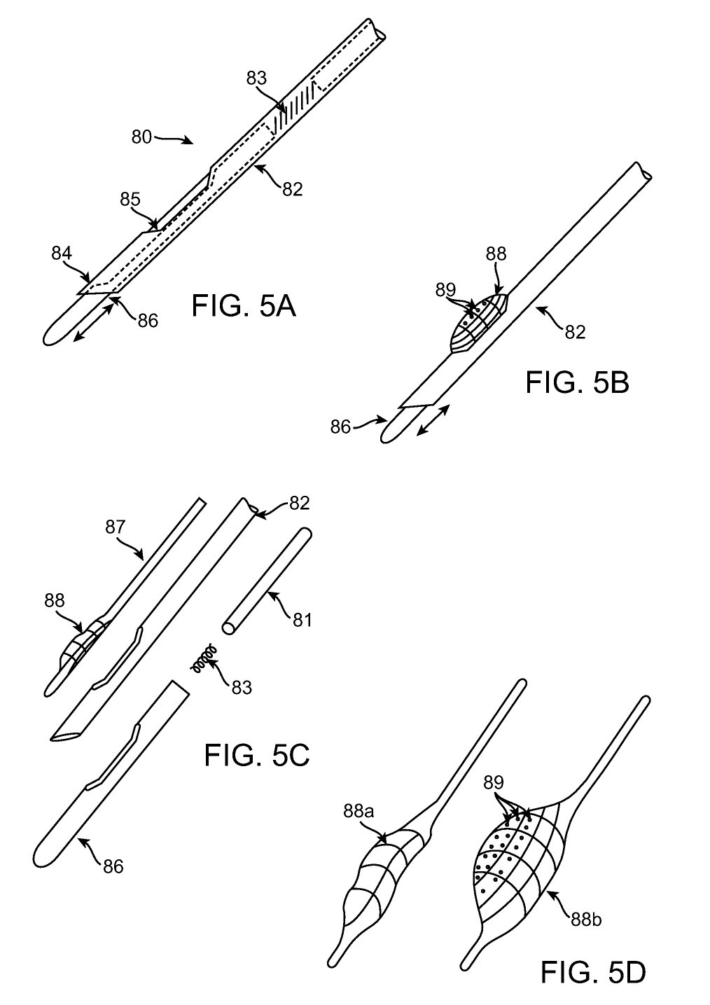

Referring now to FIGS. 5A-5D, multiple views of one embodiment of a scope cleaning device 80 are shown. FIG. 5A shows a cross-sectional view of scope cleaning device 80, including a cannula 82 with a side-wall opening 85 and a sharp distal tip 84 and a tip blunting member 86 slidably disposed in cannula 82. In this embodiment, which is similar to a varies needle, sharp distal tip 84 of cannula 82 may be used to pierce through a patient's skin and into the abdominal cavity (or another cavity), and blunting member 86 may then advance via a spring 83 beyond distal end 84 to blunt the device and prevent it from harming any internal structures within the patient.

As shown in FIG. 5B, in one embodiment, an expandable balloon 88 (or alternatively another expandable member) may be expanded out of opening 85 of cannula 82. Balloon 88 may alternatively be semi-permeable and/or include multiple holes 89, ports or perforations. Holes 89 allow fluid used to expand balloon 88 to slowly leak from balloon 88 and thus provide cleaning fluid to balloon's 88 outer surface. FIG. 5C shows an exploded view of this embodiment, including balloon 88, balloon inflation tube 87, cannula 82, blunting member 86, spring 83, and proximal spring stop 81. In one embodiment, balloon 88 and inflation tube 87 may reside within blunting member 86, spring 83 and spring stop 81, with the latter three members residing within cannula 82.

FIG. 5D shows balloon 88 in an unexpanded state 88a and expanded state 88b. In various embodiments, balloon 88 may have any suitable size and may include any number of holes 89.

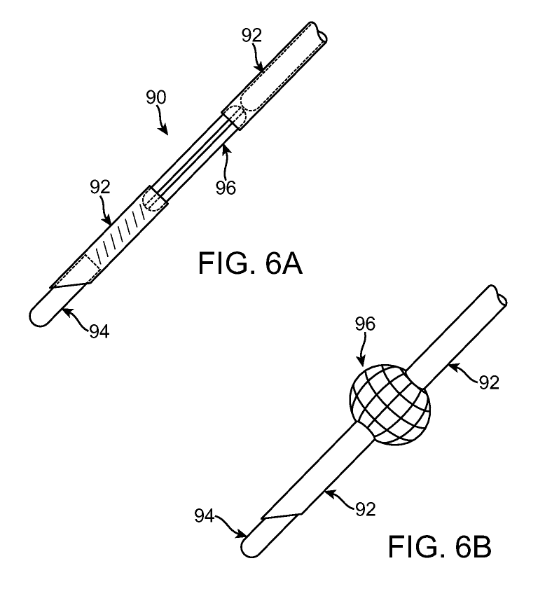

With reference now to FIGS. 6A and 6B, in an alternative embodiment, an endoscope cleaning device 90 may include a cannula 92, a tip blunting member 94, and an expandable member 96. In some embodiments, such as the one depicted in FIGS. 6A and 6B, expandable member 92 may be disposed more proximally along cannula 92 than in other embodiments. As shown in this embodiment, cannula 92 may be divided into proximal and distal portions, with expandable member 96 residing between the two.

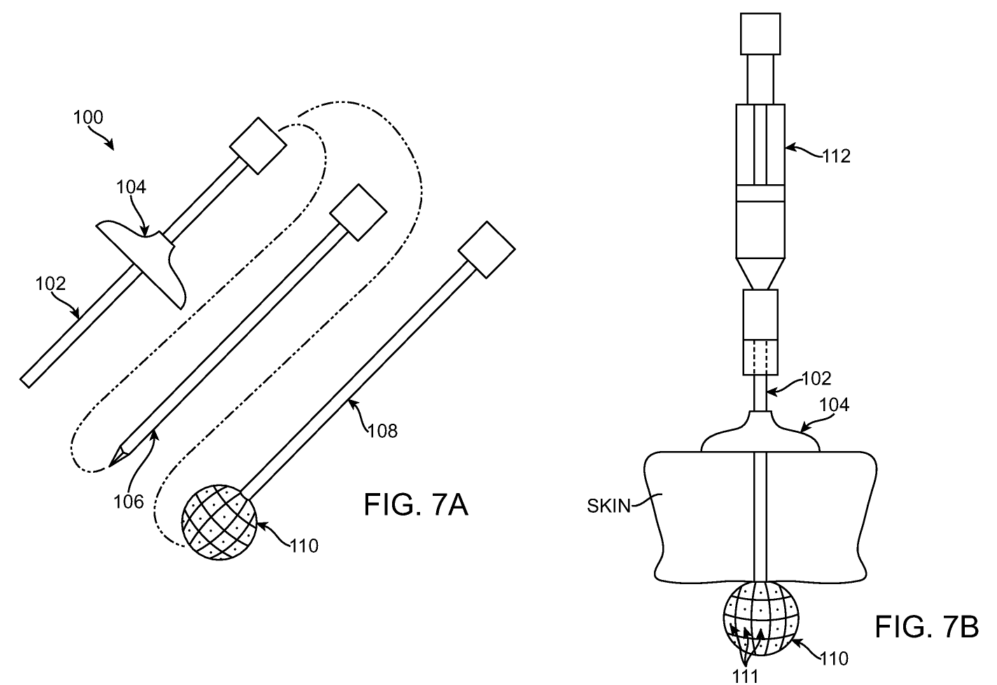

FIGS. 7A and 7B show another embodiment of an endoscope cleaning system 100. In this embodiment, cleaning system 100 may include a cannula 102 with some type of tissue anchoring member 104, a piercing member 106 which may be slidably disposed through cannula 102, and a lens cleaning member 108 which may include an expandable member 110 at or near its distal end and which may also be slidably disposed through cannula 102. As shown in FIG. 7B, in one embodiment, cannula 102 may be disposed through a patient's skin, using piercing member 106, which may then be removed and replaced with cleaning member 108. Fluid may be introduced into expanding member 110 via a syringe 112 (or alternatively any other suitable fluid introduction device), such that it slowly weeps or leaks through holes 111 to coat an outer surface of expandable member 110. In one embodiment, expandable member 110 may be disposed such that it contact an inner surface of a body cavity of a patient, and anchoring member 104 may be moved to contact an outer surface of the body cavity, such as the skin, thus effectively locking system 100 in place relative to the patient. This locking function may not completely immobilize system 100 but will likely reduce movement, so that when an endoscope is placed in contact with expandable member 110 it can exert enough force to clean the lens of the endoscope. In various alternative embodiments, anchoring member 104 may be replaced with any other suitable anchoring or anti-motion devices.

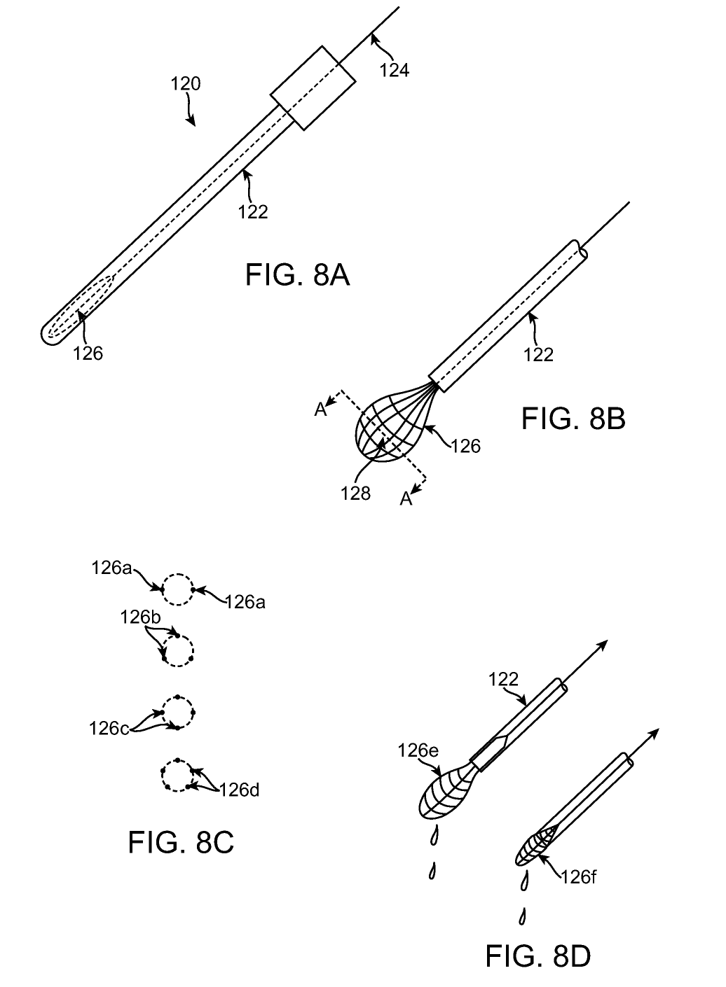

Referring now to FIGS. 8A-8D, another embodiment of an endoscope cleaning device 120 is shown. FIG. 8A shows device 120 including a cannula 122 and an expandable nitinol wire cage 126 (or “mesh,” “housing” or the like) coupled with a wire 124. In FIG. 8A, wire cage 126 is collapsed and resides in cannula 122. In FIG. 8B, wire cage 126 is expanded and extended out of cannula 122. In some embodiments, wire cage 126 may contain a sponge or other fluid retaining material 128, such as a cloth or the like, such that when fluid is introduced into material 128, material 128 expands and wire cage 126 expands. In use, an endoscope lens may be cleaned, for example, by pressing and/or wiping the lens against the expanded material 128. The fluid in material 128 may also help clean the lens.

FIG. 8C shows different alternative embodiments of nitinol wire cage 126 in end-on views. In some embodiments, for example, there may be two longitudinal wires 126a, three longitudinal wires 126b, four longitudinal wires 126c or five longitudinal wires 126d. In other alternative embodiments, wire cage 126 may include any other suitable number of longitudinal wires. Proximal wire 124 may be used in some embodiments to both push wire cage 126 out of cannula 122 and pull it back into cannula 122. As shown in FIG. 8D, fluid may leak or weep out of material 128 with cage 126e in its expanded state and/or in its collapsed state 126f. In some embodiments, fluid is squeezed out of material 128 as nitinol wire cage 126f is pulled back into cannula 122, thus allowing material to shrink to fit back into cannula 122.

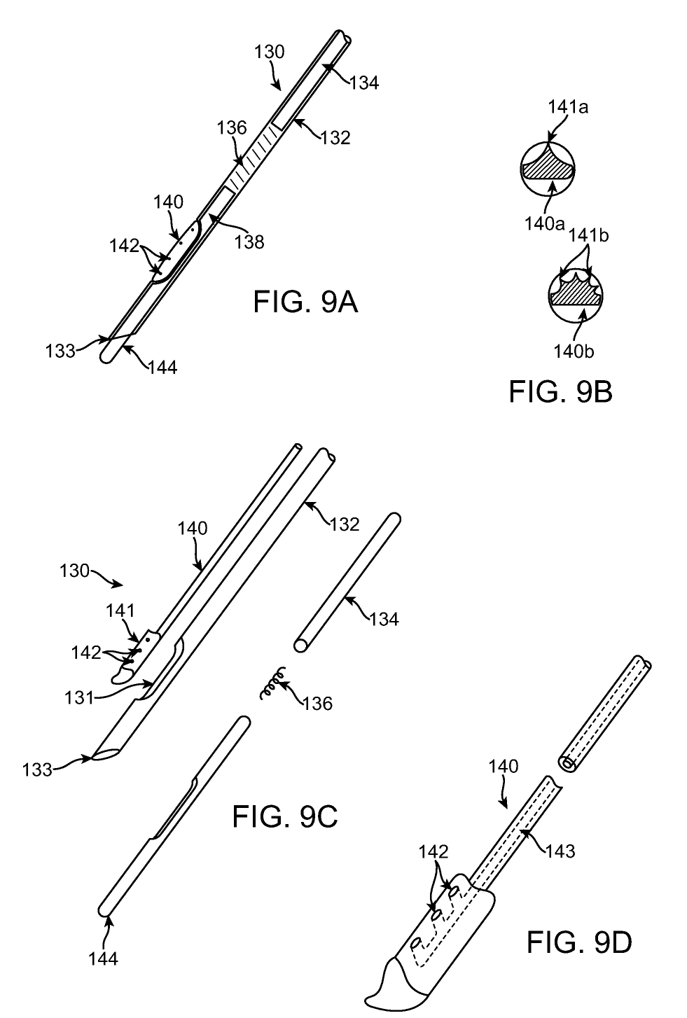

Turning to FIGS. 9A-9D, another embodiment of an endoscope lens cleaning device 130 is shown. Cleaning device 130 may suitably include a cannula 132 with a side-facing opening and a sharp distal tip 133, a proximal spring stop 134, a spring 136, a blunting member 144, and a lens cleaning member 140 including one or more wiper blades and multiple fluid irrigation ports 142. FIG. 9B shows end-on views of two different embodiments of cleaning members 140a, 140b with one wiper blade 141a and multiple wiper blades 141b. In various embodiments, any suitable number of wiper blades 141 may be included. In alternative embodiments, cleaning member 140 may be slidably disposed in cannula 132 and/or may be capable of rotating within cannula 132. Alternatively, cleaning member 140 may be fixedly disposed in cannula 132. In use, an endoscope lens may be cleaned by wiping it across one or more wiper blades 141 and/or through contact with cleaning fluid delivered through ports 142.

FIG. 9C shows an exploded view of the various components of scope cleaning device 130. FIG. 9D shows a detailed view of lens cleaning member 140, including a fluid lumen 143 in communication with ports 142. In alternative embodiments, cleaning member 140 may include any suitable number of ports 142 ports disposed at any suitable locations along cleaning member.

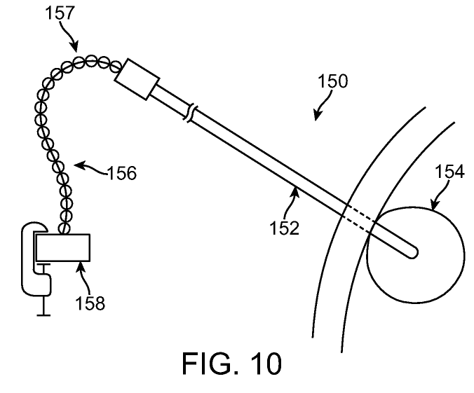

Referring now to FIG. 10, in one embodiment an endoscope lens cleaning system 150 may include a cannula 152, an expandable lens cleaning member 154 and a locking system 156. Locking system 156, for example, may include a flexible locking cable 157 and an attachment member 158 for attaching system 150 to a fixed object outside the patient, such as an operating table. Locking system 156 may be used to help stabilize lens cleaning system 150 so that a user does not have to hold system 150 with his/her hand or use an assistant for this purpose during a procedure. Other locking/anchoring members have been described above, and other alternative locking/anchoring devices may be used in alternative embodiments to help make the endoscope cleaner a “hands-free” device, once it is put in a desired position.

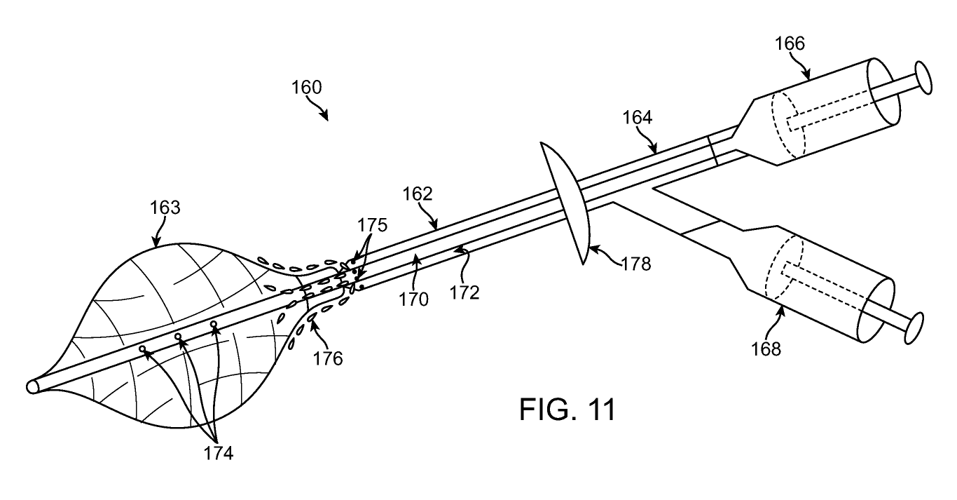

Referring now to FIG. 11, in another embodiment an endoscope cleaning device 160 may include a shaft 162 coupled with a balloon 163. Shaft 162 may include a proximal bifurcation 164 into a Y shape, with one branch connecting to a balloon inflation syringe 166 and the other to a cleaning fluid syringe 168 (alternatively, other inflation devices may be used). Shaft 162 may also include an inner lumen 170 in fluid communication with balloon inflation syringe 166 and an outer lumen in fluid communication with cleaning fluid syringe 168. Inner lumen 170 may communicate with multiple balloon inflation ports 174 (or “holes,” “apertures” or the like), for providing expansion fluid to balloon 163. Outer lumen 172 may communicate with multiple cleaning fluid apertures 175 to provide cleaning fluid 176 to the outer surface of balloon 163. Device 160 may optionally also include a stop 178, which may slide along shaft 162 to help support/stabilize device 160 against a patient during use. The dual-lumen configuration of device 160 may help keep balloon 163 inflated while also supplying cleaning fluid 176 to its outer surface.

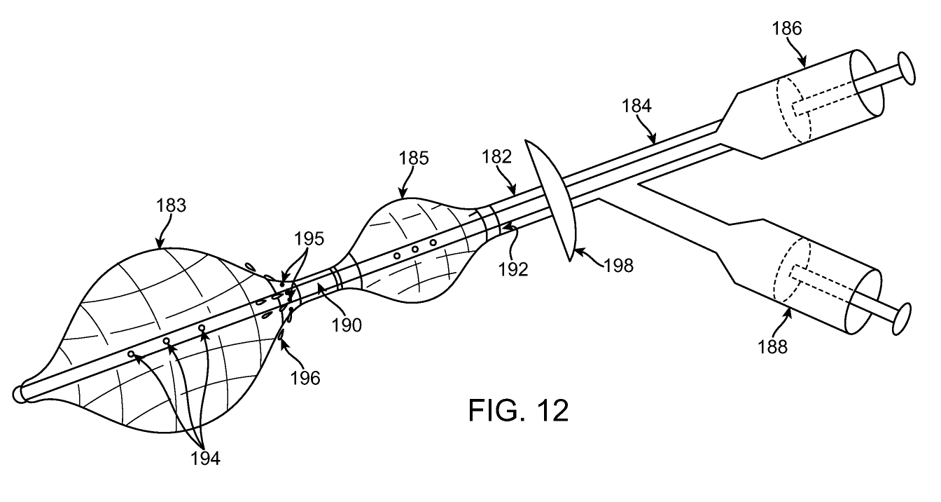

With reference now to FIG. 12, in an alternative embodiment an endoscope cleaning device 180 may include a shaft 182 with proximal bifurcation 184, an inner lumen 190, an outer lumen 192, inflation ports 194, cleaning fluid ports 195 for cleaning fluid 196, a balloon syringe 186, a cleaning syringe 188, a slidable stop 198, a distal balloon 183 and a proximal balloon 185. In this embodiment, proximal balloon 185 and stop 198 may be used together to stabilize device 180 during use, and balloon 183 with cleaning fluid 196 may be used for cleaning the endoscope lens. In this and the previous embodiments, any of a number of fluid introduction devices may be substituted on the proximal end of shaft 182, such as but not limited to dual-lumen syringes. Furthermore, shaft 182 may have any suitable proximal configuration, such as ending in a Y-shaped Luer connector or the like.

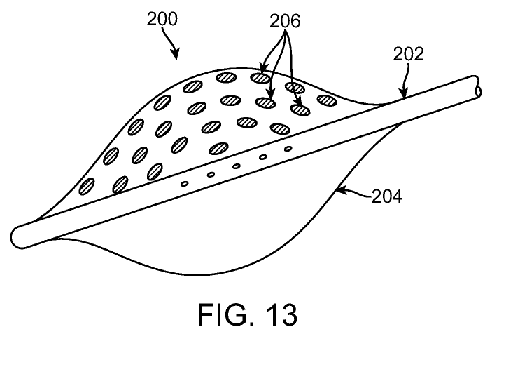

Referring now to FIG. 13, in one embodiment a distal end of an endoscope cleaning device 200 may include a shaft 202, a balloon 204 coupled with shaft 202, and one or more pieces of material 206 coupled with an outer surface of balloon 204. Material 206 may be glued onto the outer surface, incorporated into the material used to make balloon 204, formed using a mask technique or the like. Material 206 may be cotton, polyester, or any other material having absorbing ability. The absorbing material 206 acts to hold moisture, thus improving the ability of material 206 to clean an endoscope lens. In some embodiments, material 206 may cover an entire surface of balloon 204, while in other embodiments it may cover only a portion. Material 206 may be in one large piece or in multiple pieces.

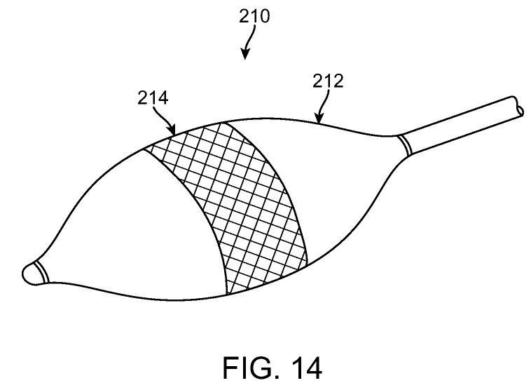

Referring now to FIG. 14, in another embodiment an endoscope cleaning device 210 may include a balloon 212 with a circumferential strip of fabric 214 disposed on its surface. As mentioned above, fabric 214 may be any suitable material and may be disposed over any part of balloon 212 in various alternative embodiments. Fabric 214 generally facilitates cleaning of an endoscope lens by providing a surface having friction across which the lens may be wiped.

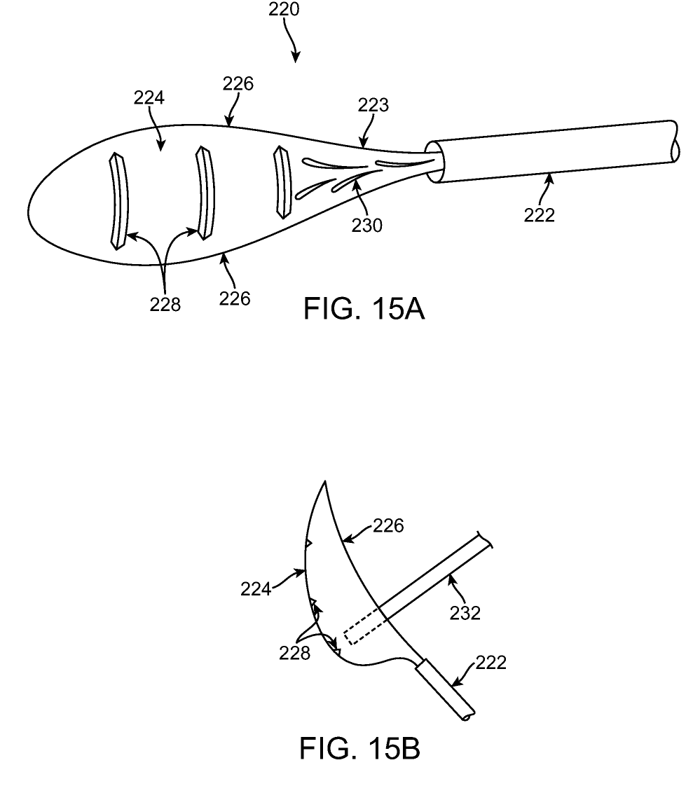

With reference now to FIGS. 15A and 15B, in yet another embodiment an endoscope cleaner 220 may include a shaft 222 and an expandable cleaning member 223 that extends out of a distal end of shaft 222. Cleaning member 223 may include an expanding Nitinol wire frame 226 with material 224 stretched across it and multiple ridges 228 built into material 224. Ridges 228, like the fabric described in previous figures, help provide frictional surfaces against which an endoscope lens may be wiped clean. Fluid 230 may be injected onto cleaning member 223 via a lumen in shaft 222. In various embodiments, any number and configuration of Nitinol wires 226 may be used, and any type and configuration of material 224 may be used.

In one embodiment, as shown in FIG. 15B, cleaning member 223 may be shaped as a sail in cross-section, thus giving more surface area against which an endoscope 232 may be cleaned. It may also be easier to press endoscope 232 into a concave structure as shown in FIG. 15B. Of course, in alternative embodiments any of a number of different configurations may be used. Cleaner 220 is configured such that Nitinol frame 226 expands when it is pushed out of the distal end of shaft 222 and collapses when it is pulled back into shaft 222. In alternative embodiments, shape memory materials other than Nitinol may be used, such as spring stainless steel and the like.

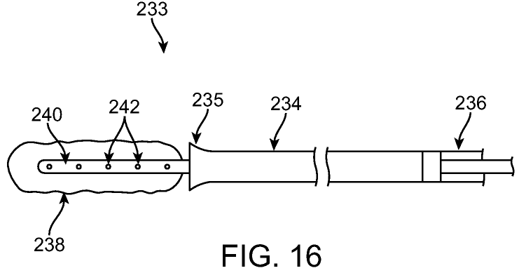

Referring now to FIG. 16, in another embodiment an endoscope cleaning device 233 may include a shaft 234 having a widened distal end 235 and a proximal end connectable to a syringe 236 or other fluid delivery device. Cleaner 233 may also include an inner shaft 240 that slidably extends out of an back into outer shaft 234 and that is attached over a distal portion to a sponge 238 for cleaning an endoscope lens. Inner shaft 240 may include one or more fluid introduction ports 242 for injecting fluid into sponge 238. Widened distal end 235 of outer shaft 234 may facilitate pulling a fluid-filled, expanded sponge 238 back into outer shaft 234. Retracting sponge 238 in this way may wring out fluid from sponge 238, thus facilitating the retraction.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

What is claimed is:

1. A device for cleaning a lens of an endoscope inside a patient during a surgical procedure, the device comprising:
a cannula having a proximal end, a distal end and an opening; and
an expandable member coupled with the cannula such that when fluid is introduced into the expandable member, it expands out of the opening in the cannula.

2. A device as in claim 1, wherein the cannula has a sharp distal tip for piercing through skin, and wherein the sharp distal tip is convertible into a blunt tip after it pierces through the skin.

3. A device as in claim 2, wherein the cannula comprises a varies needle.

4. A device as in claim 1, further including a piercing member disposed in the cannula and having a sharp tip for piercing through skin, wherein the piercing member is slidably removable from the cannula.

5. A device as in claim 1, wherein the cannula has an outer diameter of 5 mm or less.

6. A device as in claim 1, further comprising a skin anchor member slidably coupled with an outer surface of the cannula for anchoring the cannula to the patient's skin during the surgical procedure.

7. A device as in claim 1, wherein the expandable member comprises a balloon.

8. A device as in claim 7, wherein the balloon is at least semi-permeable.

9. A device as in claim 7, wherein the balloon comprises multiple pores for allowing leakage of inflation fluid onto an outer surface of the balloon.

10. A device as in claim 7, further comprising a tube coupled with the balloon and extending through the cannula to the proximal end of the cannula to provide a means for delivering expansion fluid to the balloon.

11. A device as in claim 1, wherein the expandable member comprises a fluid absorbing material.

12. A device as in claim 11, wherein the expandable member further comprises a nitinol wire cage disposed over at least part of the fluid absorbing material.

13. A device as in claim 1, wherein the opening at or near the distal end of the cannula comprises an opening in a side wall of the cannula.

14. A device as in claim 1, wherein an outer surface of the expandable member has a white color to facilitate white balancing of an endoscope device in situ.

15. A device as in claim 14, wherein the outer surface further comprises at least one of letters or symbols to facilitate focusing of the endoscope device in situ.

16. A system for cleaning a lens of an endoscope inside a patient during a surgical procedure, the system comprising:
a cannula having a proximal end, a distal end and an opening;
an expandable member coupled with the cannula such that when fluid is introduced into the expandable member it expands out of the opening in the cannula;
a fluid delivery tube coupled with the expandable member and extending through the cannula to an opening at or near the proximal end of the cannula; and
an amount of cleaning fluid for passing through the fluid delivery tube and expanding the expandable member and for cleaning the endoscope lens.

17. A system as in claim 16, further comprising a syringe, wherein the cleaning fluid is housed in the syringe.

18. A system as in claim 16, further comprising a piercing member slidably disposed within the cannula and having a sharp distal tip.

19. A system as in claim 16, wherein the fluid is selected from the group consisting of saline solution, glycol solution, water, alcohol, contact lens cleaning solution and combinations thereof.

20. A system as in claim 16, wherein the expandable member comprises a balloon, and wherein the balloon comprises multiple pores through which the cleaning fluid leaks from inside the balloon to an outer surface of the balloon.

21. A method for cleaning a lens of an endoscope inside a patient during a surgical procedure, the method comprising:
advancing a cannula into the patient;
passing fluid into an expandable member coupled with the cannula to expand the expandable member, wherein some of the fluid passes through the expandable member to an outer surface of the expandable member; and
contacting the endoscope lens with the outer surface of the expandable member, located inside the patient, to clean the lens.

22. A method as in claim 21, wherein passing the fluid into the expandable member comprises passing fluid through an inflation tube into a balloon.

23. A method as in claim 21, wherein advancing the needle comprises:
advancing the needle through skin of the patient using a sharp distal tip; and
converting the sharp distal tip to a blunt tip.

24. A method as in claim 21, further comprising anchoring the cannula to the patient's skin using a skin anchor coupled with the cannula.

25. A method as in claim 21, further comprising white balancing the endoscope inside the patient, during the procedure, using a white-colored outer surface of the expandable member.

26. A method as in claim 25, further comprising focusing the endoscope inside the patient, during the procedure, using at least one of symbols or letters disposed on an outer surface of the balloon.
